(19) United States
(12) Patent Application Publication
(10) Pub. No.: US 2015/0111304 A1
(43) Pub. Date: Apr. 23, 2015

(54) CARBON ANALYSIS USING FERRATE OXIDATION

(71) Applicant: Hach Company, Loveland, CO (US)

(72) Inventors: Richard E. Leggett, Dickinson, TX (US); John Lee, Fort Collins, CO (US); Vishnu V. Rajasekharan, Fort Collins, CO (US)

(21) Appl. No.: 14/521,626

(22) Filed: Oct. 23, 2014

Related U.S. Application Data
(60) Provisional application No. 61/981,671, filed on Apr. 18, 2014.
(60) Provisional application No. 61/894,906, filed on Oct. 23, 2013.

(51) Int. Cl. G01N 33/18

(52) U.S. Cl. CPC G01N 33/1846
USPC 436/146

(57) ABSTRACT

Methods for measuring total organic carbon in a water sample and total carbon in a water sample are disclosed.

### CROSS-REFERENCE TO RELATED APPLICATIONS

This application claims priority to U.S. provisional application No. 61/894,906 filed Oct. 23, 2013 and U.S. provisional application No. 61/981,671 filed Apr. 18, 2014.

### INTRODUCTION

This application relates generally to carbon analysis in an aqueous sample and, more particularly, to the measurement of carbon in an aqueous sample using a ferrate as an oxidizer for the organic components in the sample and measurement of generated carbon dioxide. Embodiments of a method disclosed herein are directed to the measurement of Total Inorganic Carbon (TIC), Total Organic Carbon (TOC) and Total Carbon (TC).

Commercial Total Organic Carbon (TOC) analyzers are used to measure the quantity of organic carbon present in a water sample, which is an indicator of water purity. Applications for TOC measurements include ultrapure water for pharmaceutical and electronics manufacturing, as well as municipal drinking water and wastewater and industrial wastewater from chemical and petrochemical plants, as examples.

Measurement of TOC relies on the conversion or oxidation of organic material in a water sample to CO₂, which can then be measured by conductivity or Nondispersive Infrared (NDIR) detection. Two common methods of oxidation include UV/Persulfate and high-temperature combustion. The UV/Persulfate method uses the combination of ultraviolet light and strong chemical oxidants, for example, sodium persulfate, to convert organic material to CO₂. High-temperature combustion uses thermal oxidation processes, often in the presence of catalysts, to convert the organic materials to CO₂. Both methods use an acid, for example, phosphoric acid, to initially remove “Total Inorganic Carbon” or TIC present in the water sample, as CO₂, prior to oxidation. Examples of commercially available TOC Analyzers include a UV/Persulfate TOC analyzer (GE/Sievers 900 Laboratory Analyzer) and a high-temperature combustion TOC analyzer (Shimadzu TOC-L). Another example is a combined catalytic ozone/base oxidizer (Hach Company Biotector B7000).

In TOC oxidative methods, an acid reagent is first added to convert the inorganic carbon in the sample (in the form of bicarbonate and carbonate anions) to gaseous CO₂. The CO₂ is removed by sparging the solution with a CO₂-free carrier gas, for example, purified nitrogen, to remove the liberated CO₂ which may then be measured as inorganic carbon (TIC). A chemical oxidant is then added to the solution to oxidize the organic carbon present in the sample to a carbonate species and, in the case of UV/persulfate oxidation, generally with the aid of ultraviolet radiation. The CO₂ is again sparged from the solution using a CO₂-free gas, which may then be measured as organic carbon (TOC). The sum of the TIC and TOC yields the Total Carbon (TC) in the sample.

### CARBON ANALYSIS USING FERRATE OXIDATION

An embodiment of a method for measuring total organic carbon in a water sample, comprises the steps of: (a) adding an inorganic acid to the sample to lower the pH of said sample to a pH less than about 4; (b) removing carbon dioxide generated from inorganic carbon present in the sample; (c) providing sufficient ferrate to the sample adjusted to a chosen pH for a sufficient period of time to oxidize the organic carbon therein; (d) acidifying the sample with an inorganic acid to a pH of less than about 4; (e) removing carbon dioxide generated from organic carbon present in the sample; and (f) measuring the carbon dioxide generated from organic carbon present in the sample, whereby the measurement of the total organic carbon present in the sample is obtained.

The method of paragraph [0006], wherein the inorganic acid is chosen from phosphoric acid, sulfuric acid, nitric acid, and hydrochloric acid.

The method of paragraph [0006], wherein the steps of removing the carbon dioxide comprise sparging the sample with a carrier gas.

The method of paragraph [0006], wherein the steps of removing the carbon dioxide comprise agitating the sample.

The method of paragraph [0006], wherein the steps of removing the carbon dioxide comprise applying ultrasonic energy to the sample.

The method of paragraph [0006], wherein the chosen pH value is between about 6 and about 12.

The method paragraph [0006], wherein the step of measuring the carbon dioxide comprises infrared absorption.

The method of paragraph [0006], wherein the step of providing sufficient ferrate to said sample for a sufficient period of time to oxidize the organic carbon therein comprises adding FeO₄²⁻ to the sample.

The method of paragraph [0006], wherein the step of providing sufficient ferrate to the sample for a sufficient period of time to oxidize the organic carbon therein comprises generating Fe(VI) and other high valence states of iron in solution.

The method of paragraph [0006], further comprising measuring total inorganic carbon after the step of removing carbon dioxide generated from inorganic carbon present in said sample.

The method of paragraph [0006], further comprising the step of adding ozone to the sample.

The method of paragraph [0016], wherein the ozone is added in the presence of ferrate.

The method of paragraph [0006], further comprising the step of irradiating said sample with UV light.

Another embodiment of a method for measuring total organic carbon in a water sample, comprises: dividing the sample into a first aliquot and a second aliquot; adding an inorganic base to the first aliquot to adjust the pH of the first aliquot to a chosen value; providing ferrate to the first aliquot in a sufficient amount for a sufficient time to oxidize the organic carbon therein; acidifying the first aliquot with an inorganic acid to a pH of less than about 4; measuring the carbon dioxide generated from total carbon present in the first aliquot; adding an inorganic acid to the second aliquot to lower the pH of the second aliquot to a value less than about 4; measuring the carbon dioxide generated from total inorganic carbon present in the second aliquot; and determining total organic carbon of the sample by subtracting the amount of measured carbon dioxide from the total inorganic carbon present in the second aliquot from the measured carbon dioxide generated from the total carbon present in the first aliquot.

The method of paragraph [0019], wherein the inorganic acid is chosen from phosphoric acid, sulfuric acid, nitric acid, and hydrochloric acid.

The method of paragraph [0019], further comprising the steps of removing the carbon dioxide from the second aliquot before said step of measuring carbon dioxide generated from total inorganic carbon present in the sample, and removing the carbon dioxide from the first aliquot before the step of measuring the carbon dioxide generated from total carbon present in the sample.

The method of paragraph [0021], wherein the steps of removing the carbon dioxide from the aliquots comprise sparging the aliquots with a CO₂-free carrier gas.

The method of paragraph [0021], wherein the steps of removing the carbon dioxide from said aliquots comprise agitating the aliquots.

The method of paragraph [0021], wherein the steps of removing the carbon dioxide from said aliquots comprise applying ultrasonic energy to the aliquots.

The method of paragraph [0019], wherein the chosen pH value is between about 6 and about 12.

The method of paragraph [0019], wherein the inorganic base is chosen from bases comprising hydroxide ions.

The method of paragraph [0026], wherein the inorganic base is chosen from sodium hydroxide, potassium hydroxide, and combinations thereof.

The method of paragraph [0019], wherein the step of measuring carbon dioxide generated from total inorganic carbon present in the second aliquot, and the step of measuring the carbon dioxide generated from total carbon present in the first aliquot comprise measuring infrared absorption.

The method of paragraph [0019], wherein the step of providing ferrate to the first aliquot in a sufficient amount for a sufficient time to oxidize the organic carbon therein comprises adding FeO₄²⁻ to the first aliquot.

The method of paragraph [0019], wherein the step of providing ferrate to the first aliquot in a sufficient amount for a sufficient time to oxidize the organic carbon therein comprises generating Fe(VI) and other high valence states of iron in solution.

The method of paragraph [0030], wherein the step of generating Fe(VI) and other high valence states of iron in solution comprises electrochemically generating Fe(VI) from lower valences of iron in solution.

The method of paragraph [0019], further comprising the step of adding ozone to said sample.

The method of paragraph [0032], wherein the ozone is added in the presence of ferrate.

The method of paragraph [0019], further comprising the step of irradiating said sample with UV light.

The method of paragraph [0019], wherein the first aliquot and said second aliquot are treated at the same time.

An embodiment of a method for measuring total carbon in a water sample, comprises the steps of: (a) providing sufficient ferrate to the sample adjusted to a chosen pH for a sufficient period of time to oxidize the total carbon therein; (b) acidifying the sample with an inorganic acid to a pH of less than about 4; (c) removing carbon dioxide generated from total carbon present in the sample; and (d) measuring the carbon dioxide generated from total carbon present in the sample, whereby the measurement of the total carbon present in the sample is obtained.

The method of paragraph [0036], further comprising the steps of adding an inorganic acid to the sample to lower the pH of said sample to a pH less than about 4; and removing carbon dioxide generated from inorganic carbon present in the sample, before the step of providing sufficient ferrate to the sample.

The method of paragraph [0036], wherein the inorganic acid is chosen from phosphoric acid, sulfuric acid, nitric acid, and hydrochloric acid.

The method of paragraph [0036], wherein the step of removing the carbon dioxide comprises sparging the sample with a CO₂-free carrier gas.

The method of paragraph [0036], wherein the step of removing the carbon dioxide comprises agitating the sample.

The method of paragraph [0036], wherein the step of removing the carbon dioxide comprises applying ultrasonic energy to the sample.

The method of paragraph [0036], wherein the chosen pH is between about 6 and about 12.

The method of paragraph [0036], wherein the step of measuring the carbon dioxide comprises infrared absorption.

The method of paragraph [0036], wherein the step of providing sufficient ferrate to the sample for a sufficient period of time to oxidize the organic carbon therein, comprises adding FeO₄²⁻ to the sample.

The method of paragraph [0036], wherein the step of providing sufficient ferrate to the sample for a sufficient period of time to oxidize the organic carbon therein comprises generating Fe(VI) and other high valence states of iron in solution.

The method of paragraph [0036], further comprising measuring total inorganic carbon after the step of removing carbon dioxide generated from total carbon present in the sample.

The method of paragraph [0036], further comprising the step of adding ozone to the sample.

The method of paragraph [0047], wherein said ozone is added in the presence of ferrate.

The method of paragraph [0036], further comprising the step of irradiating the sample with UV light.

### BRIEF DESCRIPTION OF THE DRAWINGS

In the drawings:

FIG. 1 is a schematic representation of an embodiment of the apparatus for determining Total Organic Carbon (TOC) or Total Carbon (TC) of a sample as a function of measured carbon dioxide released from the sample by a ferrate oxidant mixed therewith.

FIGS. 2A and 2B are graphs of the absorbance of carbon dioxide released from the apparatus of FIG. 1, resulting from ferrate oxidant mixed with samples containing indicated quantities of sucrose.

### DETAILED DESCRIPTION

Briefly, embodiments disclosed herein include methods for measurement of Total Organic Carbon (TOC), Total Inorganic Carbon (TIC) and Total Carbon (TC) in aqueous samples using higher valence iron compositions as oxidants. For example, the organic material in the sample is oxidized using ferrate either generated in situ or as the salt of an alkali metal, as examples, in a basic solution or in an appropriate pH buffer solution to optimize the oxidation process. Carbon dioxide generated during the oxidation process is measured using standard procedures and related to the carbon present in the sample. During the oxidation, the pH can be gradually lowered by adding a mineral acid such that optimum oxidation conditions may be achieved for different organic species. Oxidation using ferrate does not oxidize chloride ions and hence is not affected by the presence of chloride during organic compound oxidation process.

As used herein, the term “ferrate” means iron in a valence state greater than zero, including +1, +2, +3, +4, +5, and +6, unless the context clearly dictates otherwise.

The term “other high valence states of iron” means Fe(IV) and Fe(V).

The term “lower valences of iron” includes iron with a valence of 0-3, in other words, Fe, Fe⁺(Fe(I)), Fe²⁺(Fe(II)), and Fe³⁺(Fe(III)).

Monitoring of the ferrate depletion caused by oxidation of the organics may be used to determine when the carbon oxidation is complete. When the level of ferrate reaches steady state, further mineral acid is added to the sample to lower the pH of the oxidized sample to a pH of approximately 3, as an example, such that CO₂ may be liberated and subsequently sparged from the sample. Carbon dioxide may also be liberated from the sample by agitating the sample, and/or applying ultrasonic energy thereto, lowering the headspace pressure, or heating the sample, as examples. The released, gas-phase carbon dioxide is entrained in a carrier gas and directed to a CO₂ measurement device. The quantity of CO₂ produced is proportional to the oxidized carbon in the sample. Prior to the oxidation step, inorganic carbon may be removed as TIC, and the direct, indirect or electrochemical measurement of CO₂ following oxidation then becomes a measure of the total organic carbon in the sample.

Embodiments disclosed herein provide methods for carbon analysis in samples without the use of toxic reagents, and with high immunity to chloride ion interference. Additionally detection of ferrate depletion permits determination of completion of oxidation of the organic species in the sample.

The oxidation of the organic species by ferrate may be assisted by introducing ozone into the sample along with the ferrate, and/or applying ultraviolet radiation to the sample during the oxidation process. The production of hydroxyl radicals using ozone is described in U.S. Pat. No. 6,623,974 B1 for “Method And Apparatus For The Analysis Of A Liquid Carrying A Suspension Of Organic Matter,” the contents of which are hereby incorporated by reference herein for all that it discloses and teaches.

Sources of ferrate ion include alkali metal salts such as Na₂FeO₄ (sodium ferrate) and K₂FeO₄ (potassium ferrate). Iron in the lower valences 0-3 can be oxidized to the higher valences that have sufficient oxidation potential to oxidize organic compounds found in the environment. For example, oxidants such as ozone, hypochlorous acid and hydrogen peroxide, among others, may oxidize the lower valence forms of iron to the higher valences. Ferrate(VI) salts may be generated by oxidizing iron in an aqueous medium with strong oxidizing agents under alkaline conditions, or in the solid state by heating a mixture of iron filings and powdered potassium nitrate (R. K. Sharma (2007), Text Book Of Coordination Chemistry, Discovery Publishing House, pp. 124-125). For example, ferrates are produced by heating iron(III) hydroxide with sodium hypochlorite in alkaline solution:

2Fe(OH)₃+3OCI⁻+4OH⁻→2[FeO₄]²⁻+5H₂O+3CI⁻

(See, e.g., Gary Wulfsberg (1991), Principles of descriptive inorganic chemistry, University Science Books, pp. 142-143). The anion is typically precipitated as the barium(II) salt, forming barium ferrate. Id.

Ferrate may also be prepared in situ by electrolysis, using an iron anode and a cathode, between which a suitable electrical current is applied, and a light-transparent sample cell for measuring the generated ferrate by colorimetric detection (aqueous ferrate has its maximum absorption at approximately 504 nm). The [FeO₄]²⁻ ions are generated at the anode. An amount of alkali metal hydroxide (potassium hydroxide and/or sodium hydroxide, as examples) may be added to the sample cell to bring the cell contents to an alkaline condition. A U.S. application and a PCT application, both entitled “Apparatus, Composition and Method for Determination of Chemical Oxidation Demand” and filed concurrently herewith, describe with more particularity a system and method for generating ferrate and are incorporated by reference in their entirety herein. See also, U.S. Pat. No. 8,449,756 B2 “Method for Producing Ferrate (V) and/or (VI)”, which describes the generation of ferrate in an electrochemical cell, which is hereby incorporated by reference herein for all that it discloses and teaches.

The ferrate anion is unstable at neutral or acidic pH values, decomposing to iron(III):

[FeO₄]²⁻+3e−+8H⁺ [symbol] Fe³⁺+4H₂O.

The reduction goes through intermediate species in which iron has oxidation states +5 and +4 (See. e.g., Egon Wiberg; Nils Wiberg; Arnold Frederick Holleman (2001), Inorganic chemistry, Academic Press, pp. 1457-1458). These anions are more reactive than Fe(VI) (See, e.g., Gary M. Brittenham (1994), Raymond J. Bergeron, ed., The Development of Iron Chelators for Clinical Use, CRC Press, pp. 37-38). In alkaline conditions, ferrates are more stable, lasting for about 5 h to about 50 h at pH ≧9. Id.

Aqueous solutions of ferrates are pink when dilute, and deep red or purple at higher concentrations. The ferrate ion is a stronger oxidizing agent than permanganate (See, e.g., Kenneth Malcolm Mackay; Rosemary Ann Mackay; W. Henderson (2002), Introduction to modern inorganic chemistry (6th ed.), CRC Press, pp. 334-335), and will oxidize chromium(III) to dichromate, (See, e.g., Amit Arora (2005), Text Book Of Inorganic Chemistry, Discovery Publishing House, pp. 691-692) and ammonia to molecular nitrogen (See, e.g., Karlis Svanks (June 1976), “Oxidation of Ammonia in Water by Ferrates (VI) and (IV)” (PDF), Water Resources Center, Ohio State University, p. 3, retrieved 2013-09-30).

It is known that for acidic pH values (below about 6) ferrate oxidization of water predominates. As the pH is increased, a transition occurs where ferrate oxidation of organics begins (generally between about pH 6 to about pH 9). At above about pH 9, ferrate preferentially oxidizes organics as opposed to water.

Carbon analysis in liquid samples using ferrate oxidation may be performed under both flowing and static sample conditions.

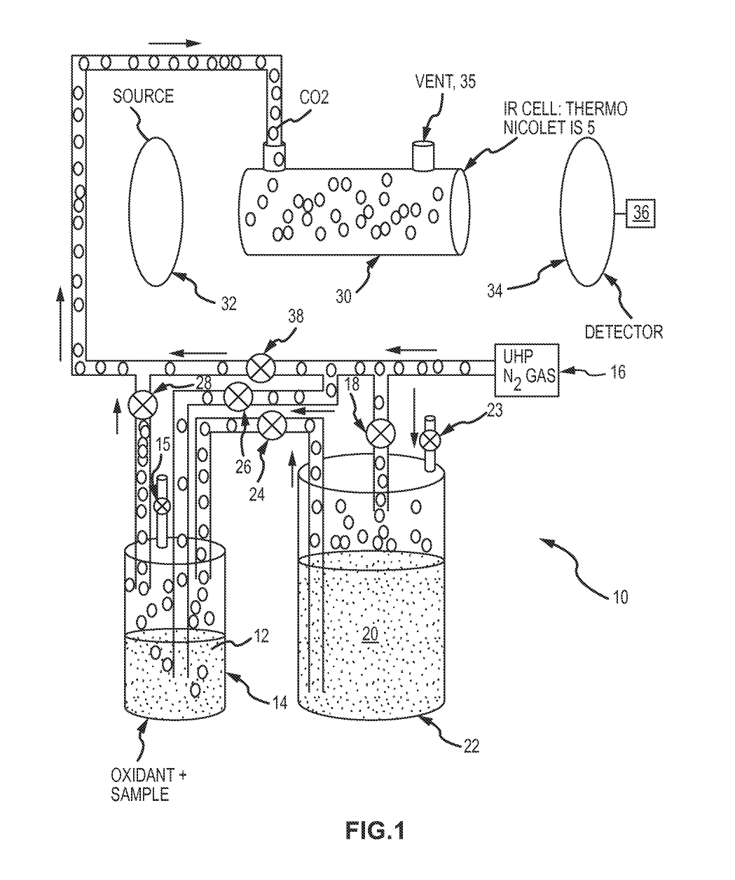

Reference will now be made in detail to exemplary embodiments illustrated in the accompanying drawings. In the FIGURES, similar structure will be identified using identical reference characters. It will be understood that the FIGURES are for the purpose of describing particular embodiments and are not intended as limiting. Turning now to FIG. 1, illustrated is a schematic representation of an embodiment of apparatus, 10, for determining Total Organic Carbon (TOC) or Total Carbon (TC) of a sample as a function of measured carbon dioxide released from the sample by a ferrate oxidant mixed therewith. A sample and ferrate oxidant, 12, are introduced into closed reaction cell, 14, having vent valve, 15, and mixed. Depending on the pH of the mixture, a base may be added to cell 14, but in any event, the pH is adjusted to be in the alkaline range. After a chosen period of time, ultrapure nitrogen or other CO₂-free gas from source, 16, directed through valve, 18, is used to drive a chosen quantity of acid, 20, such as HCl, phosphoric acid, or other mineral acid, from closed tank, 22, having vent valve, 23, through valve, 24, into cell 14 to acidify mixture 12 therein. Nitrogen or other CO₂-free sparging gas is introduced into reaction cell 14 through valve, 26, to drive carbon dioxide formed in the oxidation process and liberated from mixture 12 in reaction cell 14 through initially closed, but now open, valve, 28, into flow-through infrared absorption cell, 30, having source, 32, and detector, 34 (using the CO₂ asymmetric stretching wavelength at ˜2350 cm⁻¹), and vent, 35. Detector 34 is placed in electrical communication with electronics, 36, for determining the quantity of carbon dioxide flowing through absorption cell 30, from which the TC of the sample is determined. If the TOC is to be determined, acid 20 would be added to reaction cell 14 before the addition of the oxidant thereto, and the total inorganic carbon (TIC) would be determined from the measured carbon dioxide generated by the reaction of the acid with the inorganic carbon in the sample. The TOC is then calculated as the difference of the TC and the TIC. If only the TOC in the sample is required, the inorganic carbon can be driven off after it is generated in the initial step of the process by purging system (reaction cell 14 and absorption cell 30) by introducing nitrogen or other CO₂-free gas from source 16 through valves 26 and 38. Infrared absorption cell 30 may also be purged for initial and subsequent measurements by directing nitrogen or other CO₂-free gas from source 16 through valve 38.

As mentioned hereinabove, ozone and/or ultraviolet radiation may be used to augment the ferrate oxidation by introducing these oxidants into cell 14, by well-known methods (not shown in FIG. 1) as shown and described in U.S. Pat. No. 6,623,974 B1, which is hereby incorporated by reference herein for all that it discloses and teaches.

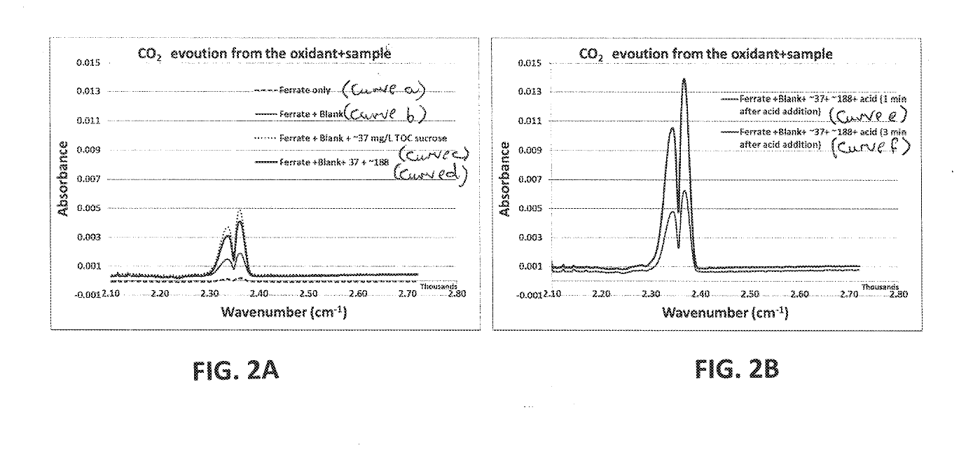

FIGS. 2A and 2B are graphs of the absorbance of carbon dioxide released from the apparatus of FIG. 1, resulting from ferrate oxidant mixed with the sample containing chosen quantities of sucrose. The stock ferrate solution comprised KOH in deionized (DI) water with approximately 10 mM K₂FeO₄ and had a pH value between approximately 9 and about 9.5. It should be mentioned that the resulting curves represent qualitative results. Curve a represents the CO₂ evolution from the ferrate oxidant (K₂FeO₄) only; Curve b, that for the ferrate oxidant plus a blank sample (high purity distilled water; no carbon present); Curve c, that for the ferrate oxidant, a blank sample and 37 mg per liter TOC of sucrose; Curve d, that for the ferrate oxidant, a blank sample and 225 mgC per liter TOC of sucrose (note that the CO₂ absorption was reduced because the addition of the larger quantity of sucrose increased the pH of the sample solution, thereby increasing the quantity of carbonate and bicarbonate formed from the generated CO₂ and remaining in solution); Curve e, that for the ferrate oxidant, a blank sample, 225 mg per liter TOC of sucrose, and acid (1 min. after the acid addition); and Curve f, that for the ferrate oxidant, a blank sample, 225 mgC per liter TOC of sucrose, and acid (3 min. after the acid addition).

Detection of CO₂ may be accomplished using an IR detector. Flow-through IR absorption cell 30 in FIG. 1 may be calibrated as follows:
1. The CO₂ absorption is first calibrated using CO₂ gas standards where the electronic output from detector 34 matches a known scaled relationship of the CO₂ that is being measured. The carrier gas flow is kept at a constant value throughout calibration and sample measurement.
2. System 10 is operated using deionized water as a blank sample plus oxidant and acid, producing minimal CO₂ concentration (C_{b}), from carbon species present in these materials. The value of C_{b} is stored.
3. A known TOC standard (S mg/L), is introduced into the reaction cell generating a CO₂ concentration of Cₛ.
4. During a sample analysis, a measured CO₂ concentration of C is generated in the absorption cell, and the TOC may be calculated from the equation:

TOC=S*(C−C_{b})/(C_{S}−C_{b}).

The foregoing description has been presented for purposes of illustration and description and is not intended to be exhaustive or limiting. Many modifications and variations are possible in light of the above teaching.

### Legend For FIG. 1

10: Measurement Apparatus
12: Sample and ferrate oxidant
14: Closed Reaction cell
15: Vent Valve
16: CO2-Free Gas Source
18: Acid Tank Pressurizing Valve
20: Acid
22: Closed Acid Tank
23: Vent Valve
24: Acid Delivery Valve
26: Sparging Gas Valve
28: Generated CO2 Release Valve
30: Absorption cell
32: IRSource
34: IR Detector
35: IR Cell Vent
36: Electronics
38: Apparatus Purge Valve

1. A method for measuring total organic carbon in a water sample, comprising the steps of:
(a) adding an inorganic acid to the sample to lower the pH of said sample to a pH less than about 4;
(b) removing carbon dioxide generated from inorganic carbon present in the sample;
(c) providing sufficient Ferrate to the sample adjusted to a chosen pH for a sufficient period of time to oxidize the organic carbon therein;
(d) acidifying the sample with an inorganic acid to a pH of less than about 4;
(e) removing carbon dioxide generated from organic carbon present in the sample; and
(f) measuring the carbon dioxide generated from organic carbon present in the sample, whereby the measurement of the total organic carbon present in the sample is obtained.

2. The method claim 1, wherein the step of measuring the carbon dioxide comprises infrared absorption.

3. The method of claim 1, wherein the step of providing sufficient Ferrate to said sample for a sufficient period of time to oxidize the organic carbon therein comprises adding FeO₄²⁻ to the sample.

4. The method of claim 1, wherein the step of providing sufficient Ferrate to the sample for a sufficient period of time to oxidize the organic carbon therein comprises generating Fe(VI) and other high valence states of iron in solution.

5. The method of claim 1, further comprising measuring total inorganic carbon after the step of removing carbon dioxide generated from inorganic carbon present in said sample.

6. The method of claim 1, further comprising the step of adding ozone to the sample.

7. The method of claim 1, further comprising the step of irradiating said sample with UV light.

8. A method for measuring total organic carbon in a water sample, comprising:
dividing the sample into a first aliquot and a second aliquot;
adding an inorganic base to the first aliquot to adjust the pH of the first aliquot to a chosen value;
providing Ferrate to the first aliquot in a sufficient amount for a sufficient time to oxidize the organic carbon therein;
acidifying the first aliquot with an inorganic acid to a pH of less than about 4;
measuring the carbon dioxide generated from total carbon present in the first aliquot;
adding an inorganic acid to the second aliquot to lower the pH of the second aliquot to a value less than about 4;
measuring the carbon dioxide generated from total inorganic carbon present in the second aliquot; and
determining total organic carbon of the sample by subtracting the amount of measured carbon dioxide from the total inorganic carbon present in the second aliquot from the measured carbon dioxide generated from the total carbon present in the first aliquot.

9. The method of claim 8, further comprising the steps of removing the carbon dioxide from the second aliquot before said step of measuring carbon dioxide generated from total inorganic carbon present in the sample, and removing the carbon dioxide from the first aliquot before the step of measuring the carbon dioxide generated from total carbon present in the sample.

10. The method of claim 8, wherein the step of measuring carbon dioxide generated from total inorganic carbon present in the second aliquot, and the step of measuring the carbon dioxide generated from total carbon present in the first aliquot comprise measuring infrared absorption.

11. The method of claim 8, wherein the step of providing Ferrate to the first aliquot in a sufficient amount for a sufficient time to oxidize the organic carbon therein comprises adding FeO₄²⁻ to the first aliquot.

12. The method of claim 8, wherein the step of providing Ferrate to the first aliquot in a sufficient amount for a sufficient time to oxidize the organic carbon therein comprises generating Fe(VI) and other high valence states of iron in solution.

13. The method of claim 8, further comprising the step of adding ozone to said sample.

14. The method of claim 8, further comprising the step of irradiating said sample with UV light.

15. A method for measuring total carbon in a water sample, comprising the steps of:
(a) providing sufficient Ferrate to the sample adjusted to a chosen pH for a sufficient period of time to oxidize the total carbon therein;
(b) acidifying the sample with an inorganic acid to a pH of less than about 4;
(c) removing carbon dioxide generated from total carbon present in the sample; and
(d) measuring the carbon dioxide generated from total carbon present in the sample, whereby the measurement of the total carbon present in the sample is obtained.

16. The method of claim 15, further comprising the steps of adding an inorganic acid to the sample to lower the pH of said sample to a pH less than about 4; and removing carbon dioxide generated from inorganic carbon present in the sample, before the step of providing sufficient Ferrate to the sample.

17. The method of claim 15, wherein the step of measuring the carbon dioxide comprises infrared absorption.

18. The method of claim 15, wherein the step of providing sufficient Ferrate to the sample for a sufficient period of time to oxidize the organic carbon therein, comprises adding FeO₄²⁻ to the sample.

19. The method of claim 15, wherein the step of providing sufficient Ferrate to the sample for a sufficient period of time to oxidize the organic carbon therein comprises generating Ferrate and other high valence states of iron in solution.

20. The method of claim 15, further comprising measuring total inorganic carbon after the step of removing carbon dioxide generated from total carbon present in the sample.
